(19) United States
(12) Patent Application Publication
(10) Pub. No.: US 2025/0032244 A1
(43) Pub. Date: Jan. 30, 2025

(54) METHOD AND ARRANGEMENT FOR PRODUCING AN OPHTHALMOLOGICAL DEVICE

(71) Applicant: Carl Zeiss Meditec AG, Jena (DE)

(72) Inventors: Michael Thaller, Oberkochen (DE); Benjamin Schreiber, Oberkochen (DE); Thorben Badur, Oberkochen (DE); Francesca Nicoli, Oberkochen (DE); Jennifer-Magdalena Masch, Oberkochen (DE); Hristina Srbinoska, Oberkocken (DE)

(21) Appl. No.: 18/786,354

(22) Filed: Jul. 26, 2024

(30) Foreign Application Priority Data
Jul. 26, 2023 (DE) 10 2023 207 117.7

(51) Int. Cl. A61F 2/16; B29C 64/106

(52) U.S. Cl. CPC A61F 2/16; B29C 64/106; A61F 2240/001

(57) ABSTRACT

A method for producing an ophthalmological device includes providing a tube transparent to electromagnetic radiation, filling the tube with a liquid which is curable with electromagnetic radiation, introducing a first component into the tube, supplying the first component to a tomographic printing apparatus with a laminar flow, creating and/or providing a data record made of images of a second component, tomographically printing the curable liquid in the tube with electromagnetic radiation using as starting point the created and/or provided data record for forming the second component, the creation and/or provision of the data record and the tomographic printing being implemented such that the second component is arranged on the first component and/or at least partially encloses the latter, and removing the first and second components arranged thereon from the at least one tomographic printing apparatus with a laminar flow. In addition, an arrangement for producing an ophthalmological device is provided.

### CROSS REFERENCE TO RELATED APPLICATIONS

This application claims priority to German patent application DE 10 2023 207 117.7, filed Jul. 26, 2023, the entire content of which is incorporated herein by reference.

### TECHNICAL FIELD

The disclosure relates to a method and an arrangement for producing an ophthalmological device.

### BACKGROUND

Ophthalmological devices, for example intraocular lenses, are conventionally produced by turning. To this end, the starting material is initially produced by polymerization. Blanks are subsequently cut from the starting material. The blanks are fastened to a turning machine, with a wax in the case of hydrophilic intraocular lenses and by being frozen on at approximately −20° C. in the case of hydrophobic intraocular lenses. A computer-controlled robotic arm equipped with a diamond tip machines the intraocular lens, for example, from the blank rotating in the turning machine. However, this is a complicated and expensive method. It is also disadvantageous that a diamond tip used during turning leaves grooves on the ophthalmological device surface, with the grooves reducing an optical quality of the ophthalmological device.

DE 10 2020 108 375 B3 describes a method for producing an intraocular lens by way of tomographic printing. The method includes the following steps: providing a container which is transparent to electromagnetic radiation and in which a liquid that is curable by the electromagnetic radiation is arranged, irradiating the liquid with a set of images formed by the electromagnetic radiation, which each depict an intraocular lens, with each of the images of the set being radiated into the liquid at a different angle of incidence with respect to a reference plane that extends through the liquid, as a result of which the liquid is cured and the cured liquid forms the intraocular lens, an actuator, a solar module and/or a sensor being arranged in the liquid and the intraocular lens being formed around the actuator, the solar module and/or the sensor.

WO 2019/043529 A1 describes a method for producing a three-dimensional object, including the calculation of a sequence of back-projections describing the three-dimensional object to be formed, said back-projections being calculated from different orientation angles of the object, the definition of a sequence of light patterns using the back-projections, and the irradiation of a photoresponsive material, which can change its material phase upon irradiation, using light with each of the light patterns at the respective corresponding orientation angle and in accordance with the defined sequence, whereby a three-dimensional distribution of changes is created within the photoresponsive medium, these changes physically reproducing the three-dimensional object, whereby the three-dimensional object is created.

### SUMMARY

It is an object of the disclosure to improve a method and an arrangement for producing an ophthalmological device.

The object is achieved by a method and an arrangement for producing an ophthalmological device as described herein.

A fundamental concept of the disclosure lies in the production of an ophthalmological device having at least one first component and at least one second component. In this case, the at least one first component is provided while, by contrast, the at least one second component is tomographically printed. As a result, the ophthalmological device can be produced in such a way that at least one second component is arranged on the at least one first component and/or at least partially encloses the latter. This allows the at least one first component to be at least partially integrated in the at least one second component. In this respect, provision is made for the at least one first component to be provided (already in its final state), with the at least one first component being able to be produced in principle with any desired production method. The at least one first component is supplied to at least one tomographic printing apparatus. This is implemented with a laminar flow in a curable liquid through a tube which is transparent to electromagnetic radiation. The at least one second component of the ophthalmological device is subsequently printed tomographically in the at least one tomographic printing apparatus. To this end, a data record made of images of the second component of the ophthalmological device is created and/or provided for the at least one second component, the images containing projections of this at least one second component from different directions. The liquid which is curable with electromagnetic radiation and contained in the transparent tube is then tomographically printed using as starting point the created and/or provided data record for forming the at least one second component, i.e., cured locally, in particular depending on the light patterns in the images of the data record. In this case, the creation and/or provision of the data record and the tomographic printing are implemented in such a way that the at least one second component is arranged on the at least one first component and/or at least partially encloses the latter. Tomographic printing of the at least one second component is followed by the at least one first component and the at least one second component arranged thereon being removed from the at least one tomographic printing apparatus with a laminar flow in the curable liquid.

In particular, a method for producing an ophthalmological device is provided, the method including: providing a tube which is transparent to electromagnetic radiation, filling the transparent tube with a liquid which is curable with electromagnetic radiation, introducing at least one first component of the ophthalmological device into the transparent tube, supplying the at least one first component to at least one tomographic printing apparatus with a laminar flow in the curable liquid, creating and/or providing a data record made of images of at least one second component of the ophthalmological device, the images containing projections of the at least one second component from different directions, tomographically printing the curable liquid in the transparent tube with electromagnetic radiation using as starting point the created and/or provided data record for forming the at least one second component with the at least one tomographic printing apparatus, the creation and/or provision of the data record and the tomographic printing being implemented in such a way that the at least one second component is arranged on the at least one first component and/or at least partially encloses the latter, and removing the at least one first component and the at least one second component arranged thereon from the at least one tomographic printing apparatus with a laminar flow in the curable liquid.

Further, an arrangement for producing an ophthalmological device in particular is created, the arrangement including a tube which is transparent to electromagnetic radiation, a flow creation apparatus configured to create a laminar flow in a liquid which is curable with electromagnetic radiation and contained in the transparent tube, a data processing apparatus, and at least one tomographic printing apparatus, the flow creation apparatus further being configured and the transparent tube being arranged to supply at least one first component of the ophthalmological device to, and remove said at least one first component from, the at least one tomographic printing apparatus with the created laminar flow, the data processing apparatus being configured to create and/or provide a data record made of images of at least one second component of the ophthalmological device, the images containing projections of this at least one second component from different directions, the tomographic printing apparatus being configured to tomographically print the curable liquid in the transparent tube with electromagnetic radiation using as starting point the created and/or provided data record for forming the at least one second component, the creation and/or provision of the data record and the tomographic printing being implemented in such a way that the at least one second component is arranged on the at least one first component and/or at least partially encloses the latter.

An advantage of the method and the arrangement is that a plurality of ophthalmological devices can be produced in the style of assembly line production. As a result, a production throughput can be increased, allowing outlay and costs to be saved overall. In this respect, provision can be made for, e.g., a plurality of first components to be moved with the laminar flow, and for these first components to be supplied to and removed from the tomographic printing apparatus. In particular, provision can be made for a multitude of simultaneous tomographic prints, wherein this can be implemented with one or more tomographic printing apparatuses. In an alternative, provision can be made for each first component to be conveyed individually into the at least one tomographic printing apparatus and for the at least one second component to be tomographically printed on an individual basis in each case for each ophthalmological device.

A further advantage of the method and the arrangement is that, in principle, any desired materials (substances) can be combined with one another. For example, provision can be made for the first component to be a haptic of an intraocular lens and for the second component to form an optical unit of the intraocular lens. A material (substance) of the first component can be chosen in virtually any way as a matter of principle and/or be produced in any desired way.

The electromagnetic radiation is in the optical wavelength range in particular, especially in the visible and/or UV wavelength range. In particular, the curable liquid may have the properties which are described in DE 10 2020 108 375 B3. In particular, tomographic printing is implemented, in principle, in the manner described in DE 10 2020 108 375 B3. For creation and/or provision purposes, the images of the data record can be calculated from for example a three-dimensional data record (e.g., CAD data) which contains the shape of the ophthalmological device, in particular the shape of the at least one second component. This is a reversed process in relation to a process used in tomographic imaging. For example, tomographic imaging is used in computed tomography. For example, tomographic imaging can make use of Radon transform. In particular, tomographic printing makes use of this reversed process in order, with the calculated images, to locally cure the curable liquid in a manner dependent on the light patterns contained in the data record images.

In particular, the curable liquid contains a solution with a dissolved monomer and a photoinitiator which can trigger a polymerization of the monomer in radiation-dependent fashion. Additionally, further substances may also be constituents of the curable liquid, for example filling substances, optically excitable dyes or nanoparticles and/or medical agents. In particular, provision is made for the curable liquid to have a specified minimum viscosity of at least 100 mPa*s (cps).

Provision can be made for the ophthalmological device to be an intraocular lens. The intraocular lens can also be an accommodating intraocular lens. The ophthalmological device, especially the intraocular lens, may in particular also include an actuator, a solar module and/or a sensor as first component. Further, the ophthalmological device, especially the intraocular lens, may include an optics body and at least one haptic.

Further, the ophthalmological device can be one of the following: a glaucoma drainage device, an eye stent, a surgical port, a capsular ring, a capsular tension ring, an eyeball ring, a capsular support device, a corneal implant, an iris implant (e.g., with dye particles), an iris prosthesis, a contact lens, a therapeutic contact lens with a medical agent, an implantable contact lens, a saddle ring, and an iris expander ring, etc.

Provision can be made for the ophthalmological device produced according to the method to be subsequently post-processed as well, for example by turning, mechanical polishing, laser polishing and/or laser cutting, etc.

Parts of the arrangement, in particular the data processing apparatus, can be deigned, either individually or together, as a combination of hardware and software, for example as program code that is executed on a microcontroller or microprocessor. However, provision may also be made for parts to be configured, either individually or together, as application-specific integrated circuits (ASICs) and/or field-programmable gate arrays (FPGAs).

In an exemplary embodiment, provision is made for the curable liquid to include a specified proportion of silica gel. For example, the proportion can be as follows without being restricted to these values: 5% (w/w), 10% (w/w), 15% (w/w), 20% (w/w), 25% (w/w), 30% (w/w), 35% (w/w), 40% (w/w), 45% (w/w), 50% (w/w), 55% (w/w), 60% (w/w). This allows a viscosity to be set. For example, provision can be made for methyl methacrylate mixed with a photoinitiator, a crosslinker (e.g., trimethylolpropane trimethacrylate) and the silica gel as thickening means to be used as curable liquid. For example, CAB-O-SIL® by Cabot Corporation can be used as silica gel.

In an exemplary embodiment, provision is made for the curable liquid to include prepolymerized methyl methacrylate and/or a mixture of long-chain polymers and short monomers. This can set, and in particular increase, a viscosity. For example, provision can be made for prepolymerized methyl methacrylate to be used in part in the mixture such that longer chains of polymethyl methacrylate (PMMA) which increase the viscosity of the curable liquid are present in part. In particular, provision is made here for complete polymerization to be prevented. Then, complete polymerization is only triggered by tomographic printing. A corresponding statement applies to the mixture of long-chain polymers and short monomers. For example, use can be made of long-chain poly(ethylene glycol) acrylate mixed with short monomers. A few representative but non-restrictive examples are listed in Table 1 below. The number of possible monomers, prepolymers, combinations of various initial materials and individual monomers is so large that only a small selection is listed, without this selection being intended to be restrictive. For example, one of the PMMA prepolymers can be combined with one or more of the secondary monomers in Table 1. It is also possible to mix various prepolymers (e.g., PMMA pre-polymers of different lengths or prepolymers of PMMA with prepolymers of the secondary monomers). Further, the mixture proportions can be varied with any possible ratio to each other in order to obtain desired properties of the resultant copolymer.

TABLE 1
Examples of formulations of PMMA prepolymers and various
copolymer candidates. In this case, each of the prepolymers
can be linked with each of the secondary
monomers. The choice of respective proportions may also vary.

| PMMA prepolymer | Secondary monomer | Crosslinker |
|---|---|---|
| (C₅O₂H₈)₅₀₋₁₀₀ | Hydroxyethyl methacrylate | Trimethylolpropane |
| (C₅O₂H₈)₁₀₀₋₂₀₀ | Ethoxyethyl methacrylate | trimethacrylate |
| (C₅O₂H₈)₅₀₀₋₇₀₀ | Ethylene glycol dimethacrylate | |
| (CO₂H₈)₈₀₀₋₁₀₀₀ | Butyl acrylate | |
| (C₅O₂H₈)₁₀₀₀₋₁₅₀₀ | Ethyl methacrylate | |

In an exemplary embodiment, provision is made for the transparent tube to be rotated about a longitudinal axis together with the curable liquid contained therein, at least during the tomographic printing. As a result, a single exposure apparatus of the tomographic printing apparatus can be used to project the respective image of the data record into the curable liquid from all directions. In this case, the viscosity of the curable liquid is chosen to be so large that, in particular, the at least one first component is rotated together with the curable liquid when the curable liquid is rotated. Provision can be made for the rotation to be started and finished with a rotational speed ramp such that a change in speed of the rotation is only small and the ability of the first component to co-rotate is ensured.

In an exemplary embodiment, provision is made for the curable liquid to be cooled with a cooling apparatus, at least in sections. As a result, a viscosity of the curable liquid can be increased, at least in sections. In particular, provision can be made for a temperature of the curable liquid to be reduced, at least during the tomographic printing, so as to prevent or at least reduce gravity-driven sinking of the at least one first component within the curable liquid during the tomographic printing.

In an exemplary embodiment, provision is made for tomographic printing of the respective at least one second component to be implemented simultaneously for a plurality of ophthalmological devices. This can increase a throughput with regards to the number of ophthalmological devices produced, whereby costs and outlay can be reduced. In this respect, provision can be made for a plurality of first components to be arranged in a tomographic printing apparatus and be tomographically printed there simultaneously, with the images of the data record to this end being created and/or provided accordingly for a plurality of second components. However, provision can also be made for a plurality of tomographic printing apparatuses, in each of which a first component or a plurality of first components are arranged for simultaneous tomographic printing of the respective second components.

In an exemplary embodiment, provision is made for a position of the at least one first component in the curable liquid to be stabilized and/or corrected with at least one energy and/or force applied to the curable liquid from the outside. In particular, this can reduce or even prevent gravity-driven sinking of the at least one first component in the curable liquid. The correction and/or stabilization can be implemented prior to the tomographic printing. However, in an alternative to that or in addition, it is also possible for the stabilization and/or correction to be performed during the tomographic printing. However, care has to be taken here that the stabilization and/or correction does not impair the tomographic printing. Provision can be made for a correction and/or stabilization apparatus to be moved parallel to the transparent tube, together with one or more first components. For example, a runner and/or a linear drive can be provided to this end. A rotational movement of the transparent tube about the longitudinal axis can also be taken into account within the scope of stabilization and/or correction. Provision can be made for a position and/or orientation of the at least one first component to be detected with at least one sensor system, with the stabilization and/or correction using the detected position and/or orientation as a starting point. For example, it is possible to determine a difference between, firstly, a detected actual position and/or actual orientation and, secondly, a target position and target orientation, respectively, and implement the stabilization and/or correction using the determined difference as a starting point.

In an exemplary embodiment, provision is made for a position of the at least one first component in the curable liquid to be stabilized and/or corrected with at least one optical tweezer. In this context, provision is made in particular for the tomographic printing not to be impaired by a wavelength used by the optical tweezer.

In an exemplary embodiment, provision is made for a position of the at least one first component in the curable liquid to be stabilized and/or corrected with at least one electric field and/or at least one magnetic field. This is possible if the first component can be influenced with electric and/or magnetic fields. In particular, directed and/or focused electric and/or magnetic fields can be created purposefully to this end. For example, if the first component contains ferromagnetic regions then the first component can be held in position with such a magnetic field.

In an exemplary embodiment, provision is made for a position of the at least one first component in the curable liquid to be stabilized and/or corrected with acousto-mechanical forces. In particular, this can be achieved with standing acoustic waves. The standing waves allow forces to be exerted on the first component, and a position of the first component can be stabilized and/or corrected in this way. For example, the method described by Adem Ozcelik et al., Acoustic tweezers for the life sciences, Nature Methods, vol. 15, issue 12, pp. 1021-1028, 26 Nov. 2018, https://doi.org/10.1038/s41592-018-0222-9, can serve as a basis.

In an exemplary embodiment, provision is made for a position of the at least one first component in the curable liquid to be stabilized and/or corrected with a laminar flow aligned counter to the gravitational force. This can hold the at least one first component in position. In particular, this exemplary embodiment can be used if the viscosity of the curable liquid is not sufficiently high to keep the at least one first component in position. By creating a laminar flow acting counter to the direction of the gravitational force, it is possible to compensate the gravity-driven sinking of the at least one first component. In this case, a speed of the laminar flow acting counter to the gravitational force is chosen in accordance with a speed at which the at least one first component sinks such that the at least one first component is held at the same position overall. Provision can be made for a position of the at least one first component to be detected with at least one sensor system, with a speed of the laminar flow acting counter to the gravitational force being selected using the detected position as a starting point. For example, a speed of sinking can be determined from a change in the detected position, and a speed of the laminar flow acting counter to the gravitational force can be selected and set using the determined speed as a starting point. In particular, the speed is chosen to have the same absolute value but a reversed sign such that a laminar flow direction is counter to the gravitational force.

Further features relating to the configuration of the arrangement arise from the description of configurations of the method. Here, the advantages of the arrangement are in each case the same as in the configurations of the method.

### BRIEF DESCRIPTION OF THE DRAWINGS

The disclosure will now be described with reference to the drawings wherein:

FIG. 1 shows a schematic flowchart of the method for producing an ophthalmological device according to an exemplary embodiment of the disclosure;

FIG. 2 shows a schematic illustration for elucidating an exemplary embodiment of the method; and

FIG. 3 shows a schematic illustration for elucidating exemplary embodiments of the arrangement.

### DESCRIPTION OF EXEMPLARY EMBODIMENTS

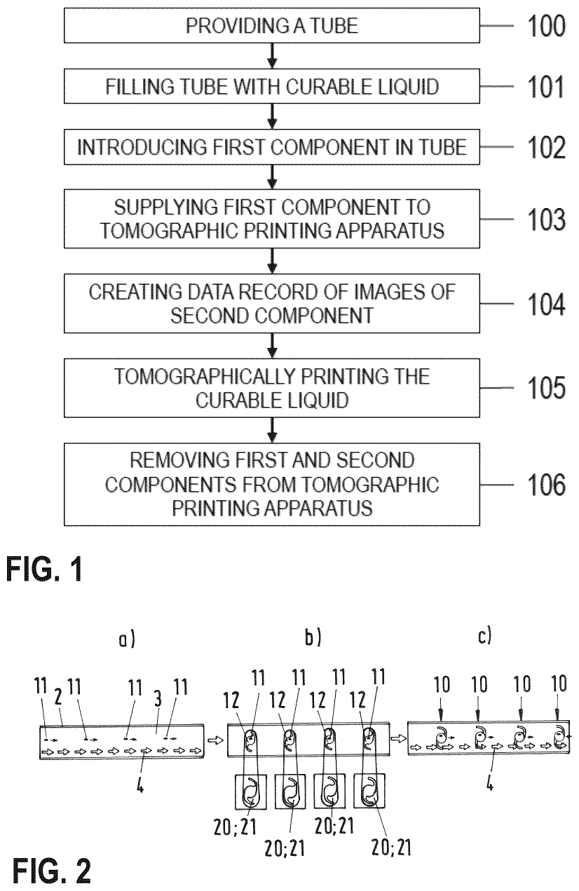

FIG. 1 shows a schematic flowchart a method for producing an ophthalmological device according to an exemplary embodiment of the disclosure. The exemplary assumption that an ophthalmological device includes a first component and a second component is made below. In principle, the ophthalmological device might also include a plurality of first components and/or a plurality of second components. For example, the ophthalmological device is an intraocular lens, with the first component being a haptic and the second component being an optical unit. The intention is to print the optical unit tomographically around a part of the haptic.

A tube which is transparent to electromagnetic radiation is provided in a method step 100. In particular, the tube extends horizontally in this case. However, the tube can also extend in a different direction as a matter of principle, for example vertically.

In a method step 101, the transparent tube is filled with a liquid which is curable with electromagnetic radiation. In this case, the curable liquid has a specified viscosity in particular.

The first component of the ophthalmological device is introduced into the transparent tube in a method step 102. In this context, the first component is arranged centrally in particular in relation to a cross-sectional area of the tube. In particular, a plurality of first components, i.e., a respective first component for a plurality of ophthalmological devices, are introduced into the transparent tube. Provision can be made for a lock to be provided to this end, with which the first component (or the plurality of first components) can be introduced into the tube and into the curable liquid.

In a method step 103, the first component is supplied to at least one tomographic printing apparatus with a laminar flow in the curable liquid. To this end, the laminar flow is created with a flow creation apparatus. In particular, in this context provision is made for a flow speed for building up the laminar flow to be modified slowly such that the first component floating in the curable liquid is moved together with the latter and does not change a relative position vis-à-vis the (moving) curable liquid but is carried along thereby.

In a method step 104, a data record made of images of the second component of the ophthalmological device is created and/or provided, the images containing projections of the second component from different directions.

In a method step 105, the curable liquid in the transparent tube is tomographically printed with electromagnetic radiation using as starting point the created and/or provided data record for forming the second component with the at least one tomographic printing apparatus. In this context, provision can be made for a speed of the laminar flow to be reduced or even set to zero to this end, with the result that the first component is moved more slowly or not at all relative to the tomographic printing apparatus while tomographic printing is taking place.

In this case, the creation and/or provision of the data record in method step 104 and the tomographic printing in method step 105 are implemented in such a way that the second component is arranged on the first component and/or at least partially encloses the latter.

In a method step 106, the first component and the second component arranged thereon are removed from the at least one tomographic printing apparatus with a laminar flow in the curable liquid.

Provision can be made for further stations to be present, to which the already produced ophthalmological device is transported with the laminar flow.

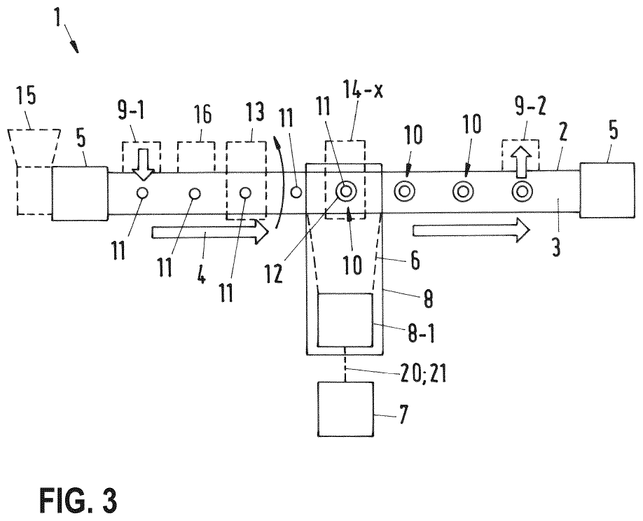

Further exemplary embodiments of the method arise from the exemplary embodiments described hereinafter with reference to FIGS. 2 and 3.

FIG. 2 shows a schematic illustration with steps a) to c) for elucidating the method. In step a), the first components 11 arranged centrally in the curable liquid 3 in the transparent tube 2 are transported to at least one tomographic printing apparatus with the laminar flow 4. In this case, the first components 11 are arranged equidistantly from one another in particular. In step b), a plurality of second components 12 are tomographically printed in such a way that the second components 12 are in each case arranged on the first component 11 and/or in each case at least partially enclose the latter. In a step c), the finished ophthalmological devices 10, which include both the first component 11 and the second component 12 arranged thereon, are removed with a laminar flow 4 and transported away from the tomographic printing apparatus as a result.

FIG. 3 shows a schematic illustration of exemplary embodiments of the arrangement 1 for producing an ophthalmological device 10. The arrangement 1 includes a tube 2 which is transparent to electromagnetic radiation 6, a flow creation apparatus 5 configured to create a laminar flow 4 in a liquid 3 which is curable with electromagnetic radiation 6 and contained in the transparent tube 2, a data processing apparatus 7, which for example includes a computing apparatus and a memory (neither of which are shown), and a tomographic printing apparatus 8.

Further, the arrangement 1 includes a lock 9-1 for loading first components 11 into the tube 2 and a lock 9-2 for unloading the finished ophthalmological devices 10 from the tube 2. The arrangement 1 may further also include a filling apparatus 15, which is configured to fill the tube 2 with the curable liquid 3.

The flow creation apparatus 5 is configured and the transparent tube 2 is arranged to supply the first components 11 of the ophthalmological device 10 to the tomographic printing apparatus 8 with the created laminar flow 4 and remove these first components from said tomographic printing apparatus following the tomographic printing.

The data processing apparatus 7 is configured to create and/or provide a data record 20 made of images 21 of a second component 12 of the ophthalmological device 10, the images 21 containing projections of the second component 12 from different directions.

The tomographic printing apparatus 8 is configured to tomographically print the curable liquid 3 in the transparent tube 2 with electromagnetic radiation 6 using as starting point the created and/or provided data record 20 for forming the second component 12. To this end, the images 21 in the data record 20 are projected into the curable liquid 3 from different directions with an exposure apparatus 8-1 of the tomographic printing apparatus 8, with the result that the curable liquid 3 is cured in targeted fashion and forms the second component 12.

In this respect, provision can be made for the transparent tube 2 to be rotated about a longitudinal axis together with the curable liquid 3 contained therein, at least during the tomographic printing, as indicated by the arrow. The arrangement 1 can have a rotation apparatus 16 to this end. Alternatively, provision can also be made for the exposure apparatus 8-1 to be rotated around the transparent tube 2 in order to provide radiation or exposure from different directions. In an alternative to that or in addition, provision can also be made for the tomographic printing apparatus 8 to include more than one exposure apparatus 8-1, with the result that exposure can be provided simultaneously from a plurality of directions.

The creation and/or provision of the data record 20 and the tomographic printing are implemented in such a way that the second component 12 is arranged on the first component 11 and/or at least partially encloses the latter, as indicated schematically in FIG. 3.

Provision can be made for the curable liquid 3 to include a specified proportion of silica gel.

Provision can be made for the curable liquid 3 to include prepolymerized methyl methacrylate and/or a mixture of long-chain polymers and short monomers.

Provision can be made for the curable liquid 3 to be cooled with a cooling apparatus 13, at least in sections. To this end, the arrangement 1 includes a cooling apparatus 13, which cools down the curable liquid 3 such that a viscosity increases. For example, the cooling apparatus 13 is arranged directly upstream of the tomographic printing apparatus 8.

Provision can be made for the tomographic printing of the second component 12 to be implemented simultaneously for a plurality of ophthalmological devices 10. To this end, a plurality of first components 11 are arranged in the tomographic printing apparatus 8 at the same time, as already shown by way of example in FIG. 2.

Provision can be made for a position of the first component 11 in the curable liquid 3 to be stabilized and/or corrected with at least one energy and/or force applied to the curable liquid 3 from the outside. To this end, the arrangement 1 includes a positioning apparatus 14-x, which performs the stabilization and/or correction. The positioning apparatus 14-x can be arranged in the tomographic printing apparatus 8, as long as it does not impair the tomographic printing procedure, and/or at another position on the transparent tube 2.

Provision can be made for a position of the first component 11 in the curable liquid 3 to be stabilized and/or corrected with at least one optical tweezer 14-1.

Further, provision can be made in an alternative to that or in addition for a position of the first component 11 in the curable liquid 3 to be stabilized and/or corrected with at least an electric field and/or at least a magnetic field, with the positioning apparatus 14-x being in the form of a field creation apparatus 14-2 to this end, the latter in particular being able to create a directed and/or focused electric and/or magnetic field.

Provision can be made in an alternative to that or in addition for a position of the first component 11 in the curable liquid 3 to be stabilized and/or corrected with acousto-mechanical forces. Then, the positioning apparatus 14-x is in the form of an acousto-mechanical modulator 14-3.

Provision can be made for a position of the first component 11 in the curable liquid 3 to be stabilized and/or corrected with a laminar flow 4 aligned counter to the gravitational force. A flow angle, for example, can be modified to this end. The tube 2 may include appropriate guiding elements (not shown), which serve to create or guide such a laminar flow 4.

In principle, provision can be made for the ophthalmological device 10 to include a plurality of first components 11 and/or a plurality of second components 12. In particular, provision can be made for further tomographic printing apparatuses to be provided in order to produce a plurality of ophthalmological devices 10 simultaneously and/or in order to tomographically print further second components separately.

### LIST OF REFERENCE NUMERALS

1 Arrangement
2 Transparent tube
3 Curable liquid
4 Laminar flow
5 Flow creation apparatus
6 Electromagnetic radiation
7 Data processing apparatus
8 Tomographic printing apparatus
8-1 Exposure apparatus
9-1 Lock
9-2 Lock
10 Ophthalmological device
11 First component
12 Second component
13 Cooling apparatus
14-x Positioning apparatus
14-1 Optical tweezer
14-2 Field creation apparatus
14-3 Acousto-mechanical modulator
15 Filling apparatus
16 Rotation apparatus
20 Data record
21 Images
100-106 Method steps

What is claimed is:

1. A method for producing an ophthalmological device, the method comprising:
providing a tube which is transparent to electromagnetic radiation;
filling the transparent tube with a liquid which is curable with electromagnetic radiation;
introducing at least one first component of the ophthalmological device into the transparent tube;
supplying the at least one first component to at least one tomographic printing apparatus with a laminar flow in the curable liquid;
creating and/or providing a data record made of images of at least one second component of the ophthalmological device, the images containing projections of the at least one second component from different directions;
tomographically printing the curable liquid in the transparent tube with electromagnetic radiation using as starting point the created and/or provided data record to form the at least one second component with the at least one tomographic printing apparatus;
the creating and/or providing of the data record and the tomographic printing being implemented such that the at least one second component is arranged on the at least one first component and/or at least partially encloses the at least one first component; and
removing the at least one first component and the at least one second component arranged thereon from the at least one tomographic printing apparatus with a laminar flow in the curable liquid.

2. The method according to claim 1, wherein the curable liquid comprises a specified proportion of silica gel.

3. The method according to claim 1, wherein the curable liquid comprises prepolymerized methyl methacrylate and/or a mixture of long-chain polymers and short monomers.

4. The method according to claim 1, wherein the transparent tube is rotated about a longitudinal axis together with the curable liquid contained therein, at least during the tomographic printing.

5. The method according to claim 1, wherein the curable liquid is cooled with a cooling apparatus, at least in sections.

6. The method according to claim 1, wherein tomographic printing of the respective at least one second component is implemented simultaneously for a plurality of ophthalmological devices.

7. The method according to claim 1, wherein a position of the at least one first component in the curable liquid is stabilized and/or corrected with at least one energy and/or force applied to the curable liquid from an outside.

8. The method according to claim 7, wherein a position of the at least one first component in the curable liquid is stabilized and/or corrected with at least one optical tweezer.

9. The method according to claim 7, wherein a position of the at least one first component in the curable liquid is stabilized and/or corrected with at least one electric field and/or at least one magnetic field.

10. The method according to claim 7, wherein a position of the at least one first component in the curable liquid is stabilized and/or corrected with acousto-mechanical forces.

11. The method according to claim 1, wherein a position of the at least one first component in the curable liquid is stabilized and/or corrected with a laminar flow aligned counter to a gravitational force.

12. An arrangement for producing an ophthalmological device, the arrangement comprising:
a tube which is transparent to electromagnetic radiation;
a flow creation apparatus configured to create a laminar flow in a liquid which is curable with electromagnetic radiation and contained in the transparent tube;
a data processing apparatus;
at least one tomographic printing apparatus;
the flow creation apparatus further being configured and the transparent tube being arranged to supply at least one first component of the ophthalmological device to, and remove the at least one first component from, the at least one tomographic printing apparatus with the created laminar flow;
the data processing apparatus being configured to create and/or provide a data record made of images of at least one second component of the ophthalmological device, the images containing projections of this at least one second component from different directions;
the tomographic printing apparatus being configured to tomographically print the curable liquid in the transparent tube with electromagnetic radiation using as starting point the created and/or provided data record for forming the at least one second component; and
creation and/or provision of the data record and the tomographic printing being implemented such that the at least one second component is arranged on the at least one first component and/or at least partially encloses the at least one first component.
